(19) United States
(12) Patent Application Publication
(10) Pub. No.: US 2025/0213516 A1
(43) Pub. Date: Jul. 3, 2025

(54) Prophylactic Methods For Treating ALS

(71) Applicant: BioJiva LLC, San Jose, CA (US)

(72) Inventors: Peter Milner, San Jose, CA (US); Nadia Litterman, San Jose, CA (US); Mark Midei, Los Altos, CA (US)

(21) Appl. No.: 18/851,852

(22) PCT Filed: Mar. 30, 2023
(86) PCT No.: PCT/US2023/016984

§ 371 Date: Sep. 27, 2024

Related U.S. Application Data
(60) Provisional application No. 63/325,464, filed on Mar. 30, 2022.

(51) Int. Cl. A61K 31/232; A61K 31/202; A61P 21/00; A61P 25/02

(52) U.S. Cl. CPC A61K 31/232; A61K 31/202; A61P 21/00; A61P 25/02

(57) ABSTRACT

Amyotrophic lateral sclerosis (ALS) is a debilitating and fatal neurodegenerative disease in humans. The underlying hallmarks of the disease involve lipid peroxidation (LPO) of polyunsaturated fatty acids (PUFAs) in the motor neurons. Central to this oxidative pathway is the presence of labile bis-allylic hydrogen atoms found in arachidonic acid, the dominant PUFA found in neurons. Disclosed are prophylactic methods for delaying the onset of the accelerated loss of functionality in patients suffering from ALS in humans. The methods comprise use of a deuterated arachidonic acid or a prodrug thereof.

### CROSS-REFERENCE

This application claims the benefit of U.S. Provisional Application No. 63/325,464, filed Mar. 30, 2022, which is hereby incorporated by reference in its entirety herein.

### TECHNICAL FIELD

Disclosed are prophylactic methods for delaying the onset of the accelerated loss of functionality in patients suffering from amyotrophic lateral sclerosis (ALS) in humans. The methods comprise use of a deuterated arachidonic acid or a prodrug thereof.

### BACKGROUND

ALS is a debilitating and fatal neurodegenerative disease in humans that, despite the best efforts of researchers, remains incurable. As such, the attending clinician attempts to slow the progression of the disease and maintain the quality of life for the patient for as long as possible.

ALS typically occurs later in life and is a neurological disease with its corresponding pathological hallmarks including progressive muscle weakness, muscle atrophy and spasticity all of which reflect the degeneration and death of upper or lower motor neurons. When diagnosed early, most patients exhibit an initial slower rate of disease progression where the extent of loss of muscular functionality is limited as compared to the accelerated loss of muscular functionality that occurs later. During this incipient phase of ALS, the patient retains significant levels of functionality even in the absence of therapy.

The incipient phase of ALS precedes an accelerated phase where the loss of muscular functionality proceeds rapidly and then terminates in death typically within 3 to 4 years from diagnosis with some patients succumbing even earlier. While the underlying cause of death is ALS, patients often succumb to pneumonia that is induced by aspiration of food into the lungs resulting from inability to swallow properly.

The underlying hallmarks of the disease involve lipid peroxidation (LPO) of polyunsaturated fatty acids (PUFAs) in the motor neurons. Central to this oxidative pathway is the presence of labile bis-allylic hydrogen atoms found in arachidonic acid, the dominant PUFA found in neurons. The structure of arachidonic acid including identification of the bis-allylic sites is as follows:

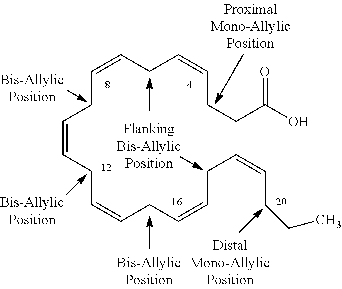 [SMILES: CCC/C=C\C/C=C\C/C=C\C/C=C\C/C=C\C/C=C\CCC(=O)O]

In cellular membranes, arachidonic acids are stacked together. Oxidative processes involving reactive oxygen species (ROS) act as an initiator for autoxidation of PUFAs, including arachidonic acid, by extraction of the bis-allylic hydrogen and formation of an oxidative reactive species in the PUFA. Initial oxidation at a first bis-allylic site then leads to serial oxidation of further PUFAs in the membrane of the cell or the mitochondria. The oxidative process starts with hydrogen extraction at a bis-allylic site on the first PUFA and proceeds in a serial manner to the next PUFA and then the next PUFA and so on. At some point, the oxidative process damages or destroys the viability of the neuron leading to furtherance of the disease condition that is responsible for generation of the excessive amounts of ROS.

Heretofore, the art has disclosed that the loss of muscular functionality in ALS can be attenuated by deuteration at one or more of the bis-allylic sites of arachidonic acid found in the neurons. The stability of the deuterium-carbon bond against such oxidative processes is significantly stronger (more stable) than that of the hydrogen-carbon bond. This means that the generation of an oxidative species at the bis-allylic sites is so reduced by the carbon-deuterium bonds that the lipid peroxidative pathway is inhibited. In turn, termination of this pathway leads to enhanced survival of the neurons and, as such, attenuates the progression of the disease.

While such treatment inhibits the rate of loss of functionality, there has been no disclosure of treating patients during the incipient stage of ALS to delay the onset of the accelerated stage of ALS thereby preserving significant muscular functionality for as long as possible.

### SUMMARY

Disclosed are methods that delay the onset of the accelerated stage of ALS or attenuates the loss of muscular functionality during the accelerated stage of the disease. If otherwise untreated, the accelerated stage of ALS results in rapidly increases the loss of functionality and eventually resulting in death of the patient. The methods are designated herein as “prophylactic” in nature as they are administered to patients during the incipient stage of ALS where the disease and the corresponding loss of muscular functionality has yet to enter the accelerated stage.

Accordingly, in one embodiment, there is provided a method to delay the onset of and/or attenuate the rate of loss of muscular functionality during the accelerated stage of amyotrophic lateral sclerosis (ALS) which stage is characterized by the rapid loss of muscular functionality in patients the method comprising:
(a) ascertaining a current level of muscular functionality in a patient prior to initiating treatment by generating a natural history score using the ALSFRS-R scoring system, wherein the patient is identified as having incipient ALS when the patient scores at least 39 points out of a total of 48 possible points; and
(b) initiating treatment by periodically administering to the patient identifed as being in the incipient stage of ALS with a prophylactic dosing regimen of a deuterated arachidonic acid or a prodrug thereof thereby delaying the onset of or attenuating the rate of loss of muscular functionality during the accelerated stage of the disease.

In one embodiment, said deuterated arachidonic acid or a prodrug thereof is 11,11-D2-linoleic acid or an ester thereof. In vivo, the ester is hydrolyzed to provide for 11,11-D2-linoleic acid. A portion of this acid is then enzymatically converted in vivo to 13,13-D2-arachidonic acid. This deuteraterd arachidonic acid is then transported into the cerebral spinal fluid where it is then taken up by the motor neurons.

In one embodiment, the prodrug is 11,11-D2-linoleic acid ethyl. This drug is administered daily to the patient at a dose of about 5 to about 10 grams/day.

In one embodiment, the patient is evaluated for uptake of 13,13-D2-arachidonic acid. Such is accomplished by assessing the concentration of 13,13-D2-arachidonic acid in red blood cells as described in International Patent Application Serial No. PCT/US2022/015368 which is incorporated herein by reference in its entirety.

A steady state concentration of about 20% of 13,13-D2-arachidonic acid is found in the red blood cells when a daily dosage of about 8.64 grams is administered to the patient over a period of about 4 to about 10 weeks. This steady state concentration is based on the total amount of deuterated arachidonic acid present therein including deuterated arachidonic acid. In an embodiment, 9 grams of 11,11-D2-linoleic acid ethyl ester is administered per day. However, a portion of 9 grams comprises the ethyl ester which is removed in vivo. In addition, there is a minor amount of impurities in the product and both of which are accounted for in the amount of drug delivered. As such, the net dosing of the 11,11-D2-linoleic acid delivered to the patient is calculated to be about 8.64 grams per day.

In one embodiment, a steady state concentration of about 20% of 13,13-D2-arachidonic acid is achieved in vivo using a dosing of about 8.64 grams per day

In one embodiment, said deuterated arachidonic acid or a prodrug thereof is 7,7,10,10,13,13-D6-arachidonic acid or an ester thereof. In vivo, the ester is hydrolyzed to provide for 7,7,10,10,13,13-D6-arachidonic acid. A portion of this acid is then transported into the cerebral spinal fluid where it is then taken up by the motor neurons.

In one embodiment, the prodrug is 7,7,10,10,13,13-D6-arachidonic acid ethyl ester. In one embodiment, this drug is administered daily at a dose of about 0.1 to about 2 grams per day. In another embodiment, this drug is administered daily to the patient at a dose of about 0.25 to about 2 grams/day.

In one embodiment, the patient is evaluated for uptake of 7,7,10,10,13,13-D6-arachidonic acid. As above, this is accomplished by assessing the concentration of 7,7,10,10,13,13-D6-arachidonic acid in red blood cells.

It is contemplated that a steady state concentration of about 6% to about 20% and preferably about 10% of 7,7,10,10,13,13-D6-arachidonic acid is found in the red blood cells when a dosage of about 1 gram per day is administered to the patient. This steady state concentration is based on the total amount of arachidonic acid present therein including deuterated arachidonic acid.

In one embodiment, the dosing regimen of the deuterated arachidonic acid or prodrug thereof employed is maintained for the 6 months from the start of therapy for those patients assigned to the incipient stage of ALS. This is because the rate of loss of muscular functionality in these patients is minimal. Without being limited to any theory, the methods herein are predicated on bolstering the cell membranes of the at-risk neurons such that when the incipient stage of the disease transitions to the accelerated stage, the at-risk neurons have been pre-treated to better withstand lipid peroxidation associated with loss of functionality. This, in turn, protects these neurons from cell damage or death which then translates into a higher level of retained functionality.

### BRIEF DESCRIPTIONS OF THE DRAWINGS

FIG. 1 illustrates the change in ALSFRS-R scores in patients in the incipient stage of ALS and treated with either 11,11-D2-linoleic acid ethyl ester or placebo over the first 24 weeks of a clinical study.

FIG. 2 illustrates the change in ALSFRS-R scores in patients in the accelerated stage of ALS and treated with either 11,11-D2-linoleic acid ethyl ester or placebo over the first 24 weeks of a clinical study.

FIG. 3A illustrates the contemplated change in ALSFRS-R scores in patients initially in the incipient stage of ALS and treated with either 11,11-D2-linoleic acid ethyl ester or placebo over weeks 25 to 48 from the start of a clinical study evidencing that loss of functionality is reduced as compared to placebo at the end of week 48.

FIG. 3B illustrates the contemplated change in ALSFRS-R scores in patients initially in the incipient stage of ALS and treated with either 11,11-D2-linoleic acid ethyl ester or placebo over weeks 25 to 48 from the start of a clinical study evidencing that the transition from the incipient stage to the accelerated stage of ALS is delayed as well as attenuating the loss of functionality. Since significant loss of functionality has not been observed during the incipient stage of ALS, a steady and consistent dosing of the deuterated arachidonic acid or a prodrug thereof is preferred.

### DETAILED DESCRIPTION

Disclosed are methods for delaying the progression of amyotrophic lateral sclerosis (ALS) in humans from the incipient stage into the accelerated stage of the disease.

Prior to discussing this invention in more detail, the following terms will first be defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 15%, 10%, 5%, 1%, or any subrange or subvalue there between. Preferably, the term “about” when referencing an amount or other feature including a dose amount, means that that amount may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein, the term “linoleic acid” refers to the compound and a pharmaceutically acceptable salt thereof having the formula provided below and having the natural abundance of deuterium (i.e., about 0.0156% naturally occurring deuterium) at each hydrogen atom:

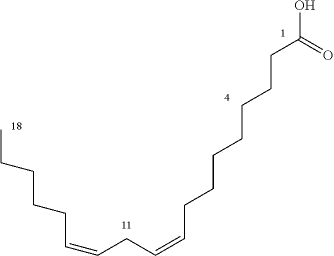 [SMILES: CCCCC/C=C\C/C=C\CCCCCCCC(=O)O]

Esters of linoleic acid are formed by replacing the —OH group with—OR. Such esters are as defined herein below.

As used herein and unless the context dictates otherwise, the term “deuterated linoleic acid or an ester thereof” refers to 11,11-D2-linoleic acid or a C₁-C₆ alkyl ester, a glycerol ester (including monoglycerides, diglycerides and triglycerides), sucrose esters, phosphate esters (e.g., phospholipids), and the like. The particular ester group employed is not critical provided that the ester group is pharmaceutically acceptable (non-toxic and biocompatible).

As used herein and unless the context dictates otherwise, the term “deuterated D2-arachidonic acid or an ester thereof” refers to 13,13-D2-arachidonic acid or a C₁-C₆ alkyl ester, a glycerol ester (including monoglycerides, diglycerides and triglycerides), sucrose esters, phosphate esters (e.g., phospholipids), and the like. The particular ester group employed is not critical provided that the ester group is pharmaceutically acceptable (non-toxic and biocompatible).

As used herein, the term “7,7,10,10,13,13-D6-arachidonic acid” includes both 7,7,10,10,13,13-D6-arachidonic acid as well as compositions of 7,7,10,10,13,13-D6-arachidonic acid that comprise, on average, at least about 80% of the hydrogen atoms at each of the bis-allylic sites having been replaced by deuterium atoms and, on average, no more than about 35% of the hydrogen atoms at the mono-allylic sites having been replaced by deuterium atoms. For example, in the case of 80% deuteration of the 3 bis-allylic sites in arachidonic acid and 35% deuteration of the mono-allylic sites, the total amount of deuteration is (6×0.8)+(4×0.35)=6.2 exclusive of the nominal amount of naturally occurring deuterium in each of the remaining methylene and methyl groups within arachidonic acid. Methods for preparing such compositions of deuterated arachidonic acid are found in U.S. Pat. No. 10,730,821 which is incorporated herein by reference in its entirety.

As used herein and unless the context dictates otherwise, the term “7,7,10,10,13,13-D6-arachidonic acid or an ester thereof” refers to C₁-C₆ alkyl esters, glycerol esters (including monoglycerides, diglycerides and triglycerides), sucrose esters, phosphate esters (e.g., phospholipids), and the like. The particular ester group employed is not critical provided that the ester group is pharmaceutically acceptable (non-toxic and biocompatible).

As used herein, the term “phospholipid” refers to any and all phospholipids that are components of the cell membrane. Included within this term are phosphatidylcholine, phosphatidylethanolamine, phosphatidylserine, and sphingomyelin. In the motor neurons, the cell membrane is enriched in phospholipids comprising arachidonic acid.

As used herein, the term “pathology of a disease” refers to the cause, development, structural/functional changes, and natural history associated with that disease. The term “natural history” means the progression of the disease in the absence of treatment per the methods described herein.

In ALS, the rate of disease progression is measured by the Revised ALS Functional Rating Scale (“ALSFRS-R”) which is found at https://www.mdcalc.com/calc/10166/revised-amyotrophic-lateral-sclerosis-functional-rating-scale-alsfrs-r which is incorporated herein by reference in its entirety.

This rating scale evaluated 12 different components on a 0 (worse) to 4 (best) scale where the components are speech, salivation, swallowing, handwriting, walking, food handling, dressing and hygiene, turning in bed, walking, climbing stairs, dyspnea, orthopnea, and respiratory insufficiency. As used herein, patients diagnosed with ALS are first evaluated to determine their natural history which measures the extent of loss of muscular functional due to the disease prior to initiation of therapy.

As used herein, the term “incipient stage of ALS” refers to patients whose disease progression has evidenced only modest loss of muscular functionality. For the purposes of this application, such is identified as having a natural history score of 39 or above.

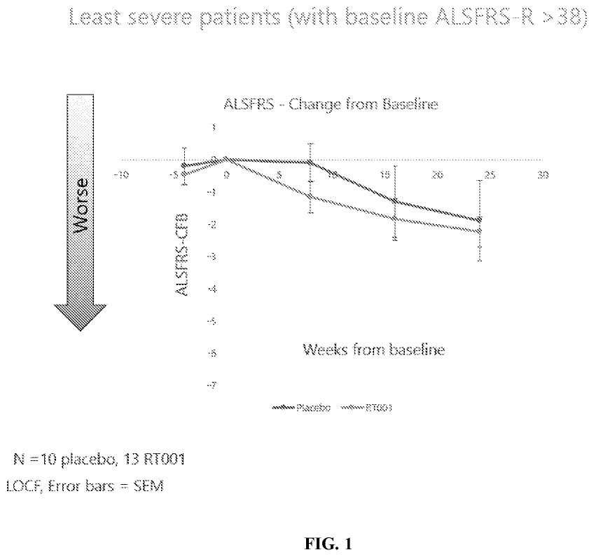

As shown in FIG. 1, patients in the incipient stage of ALS do not evidence a significant change in their ALSFRS-R scores 6 months (24 weeks) into a clinical trial regardless of whether the patients are being treated with 11,11-D2-linoleic acid ethyl ester or with placebo. Indeed, the average score for treated and untreated patients after 24 weeks is substantially identical. The data evidence that those patients in the incipient stage of ALS have yet to experience a significant loss of functionality as is found in the accelerated stage of the disease.

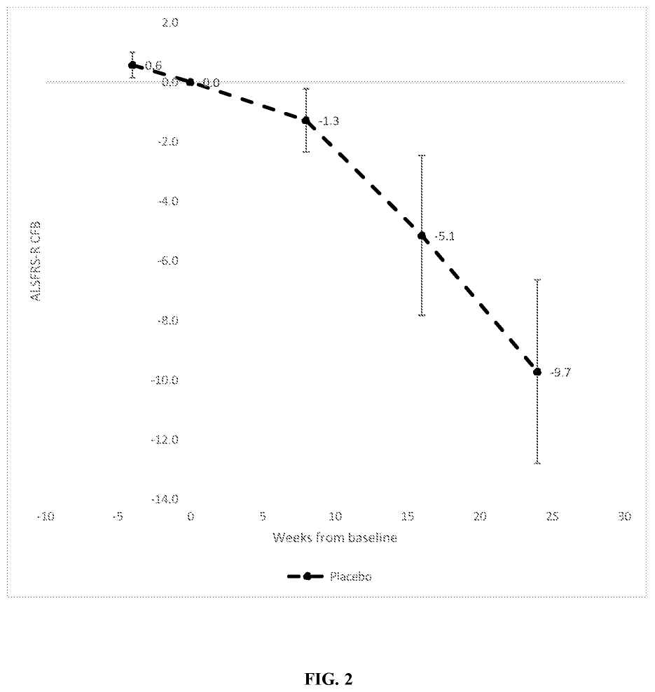

As used herein, the term “accelerated stage of ALS” refers to patients whose diseases progression has evidenced a more substantial loss of muscular functionality. For the purposes of this application, such is identified as having a natural history score of 37 or less. As shown in FIG. 2, patients in the accelerated stage of ALS and treated with placebo evidence significant loss of functionality during the first 24 weeks of the clinical trial.

In view of the above, the benefit of therapy as per the methods described herein is analyzed in patients designated to be in the incipient stage of ALS and is ascertained subsequent to the initial 24 weeks of therapy. In particular, the therapy described herein is analyzed as being prophylactic in nature. That is to say that the benefit exhibited by the therapy is measured at 48 weeks after start of therapy by comparing the net loss of functionality from 25 weeks to 48 weeks for the patients on therapy (first cohort) and those on placebo (second cohort). The benefit provided is ascertained by either a longer duration in the incipient stage of the disease for the first cohort as compared to the second cohort or by slower loss of functionality when the patients in the first cohort transition into the accelerated stage of the disease as compared to the second cohort. In either case, such benefits are ascertained by averaging the aggregate score achieved by the patients in the first and second cohorts at 48 weeks. The loss of functionality between weeks 25 to 48 weeks is measured as a delta. A benefit is perceived if the delta for the first cohort is at least about 2 points higher on average than the placebo cohort. Preferably, the delta is at least about 3 points or at least about 4 points or more.

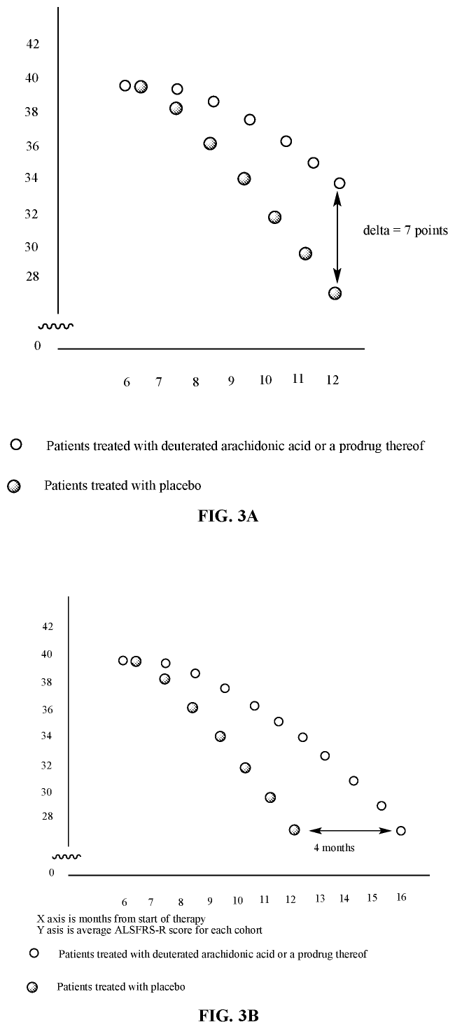

In one embodiment, the ALSFRS-R scores for each patient in both cohorts are conducted monthly, bimonthly or quarterly as well as the semi-annual scores. A representative plot showing a contemplated result is found in FIG. 3A and assumes that the patients will be scored monthly for their extent of muscular functionality in the ALSFRS-R evaluation. As shown in FIG. 3A, patients in the incipient stage of ALS do not evidence a significant change in there ASLFRS-R scores 6 months (24 weeks) after start of therapy regardless of whether the patients are being treated with 11,11-D2-linoleic acid ethyl ester or with placebo. At 48 weeks (i.e., 24 weeks after the initial 6 month score provided by the ALSFRS-R test), the average for cohort 1 is contemplated to be about a +7 higher than cohort 2. FIG. 3B illustrates the delay of 4 months in reaching the same point of loss of functionality between cohort 1 and cohort 2. A benefit is perceived if the delta for the average of the first cohort provides for at least 1 month of additional time prior to reaching the same reduced level of functionality as found with the placebo cohort and preferably at least 2 months, or at least 3 months, or at least 4 months or more.

As used herein, the term “patient” refers to a human patient or a cohort of human patients suffering from ALS.

Because ALS patients in the incipient stage of the disease show minimal changes in their functionality during the first 6 months independent regardless of whether these patients were treated as described herein or with placebo, the attending clinician is allowed some flexibility in dosing the patient provided that the patient is at a steady state concentration for the particular deuterated arachidonic acid or prodrug thereof used which is also dependent on the dosage used. In one embodiment, the clinician can prescribe a dose of 11,11-D2-linoleic acid ethyl ester that is from about 5 to about 10 grams/day. That amount is defined herein as it relates solely to treating patients in the incipient stage of the disease as a “loading or primer amount” and is intended to achieve a steady state concentration of deuterated arachidonic acid in the body as evidenced by red blood cells. As the conversion of 11,11-D2-linoleic acid to 13,13-D2-arachidonic acid in vivo coupled with uptake into neurons is a slow process, sufficient amounts of 11,11-D2-linoleic acid or an ester thereof is administered to the patient such that a steady state concentration of about 20% or more in red blood cells is achieved within about 8 weeks after the start of therapy.

As used herein, the term “maintenance dose” refers to a dose of deuterated arachidonic acid or a prodrug thereof that is less than the primer dose and is sufficient to maintain the desired steady state concentration of the deuterated arachidonic acid in red blood. That is to say that the maintenance dose can be used to maintain the desired steady state concentration in vivo without the need to accumulate more deuterated arachidonic acid into the neurons or the red blood cells.

As used herein, the term “periodic dosing” refers to a dosing schedule that substantially comports to the dosing described herein. Stated differently, periodic dosing includes a patient who is compliant at least 75 percent of the time over a 30-day period and preferably at least 80% compliant. In embodiments, the dosing schedule contains a designed pause in dosing. For example, a dosing schedule that provides dosing 6 days a week is one form of periodic dosing. Another example is allowing the patient to pause administration for from about 3 or 7 or more days, e.g., due to personal reasons, provided that the patient is otherwise at least 75 percent compliant.

As used herein, the term “cohort” refers to a group of at least 5 patients whose results are to be averaged.

As used herein, the term “pharmaceutically acceptable salts” of compounds disclosed herein are within the scope of the methods described herein and include acid or base addition salts which retain the desired pharmacological activity and is not biologically undesirable (e.g., the salt is not unduly toxic, allergenic, or irritating, and is bioavailable). When the compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt.

The phrase “excessive amounts of PUFAs,” “excessive PUFA intake,” and the like refer to intake of total PUFAs (e.g., total amount of PUFAs consumed per day) that result in reduced conversion of 11,11-D2-linoleic acid to 13,13-D2-arachidonic acid compared to a diet lower in total PUFA intake. In embodiments, the patient is on a diet that restricts intake of linoleic acid, arachidonic acid, and/or other PUFA compounds. The amount of PUF As that can be consumed by a patient is variable, depending on numerous factors such as the patient's health, weight, age, other medications being taken, liver function, metabolism, and the like.

In general, a patient on a 2,000 calorie per day diet consumes up to about 22 grams of polyunsaturated fatty acids (https://news.christianacare.org/2013/04/nutrition-numbers-revealed-fat-intake/), of which about 14 grams are linoleic acid when averaged for men and women (https://www.ncbi.nlm.nih.gov/pmc/articles/PMC3650500/). In addition, only about 10% of the average amount of linoleic acid consumed is hepatically converted to arachidonic acid. So, on average, about 1.4 grams of arachidonic acid is generated per day. When a patient consumes excessive amounts of PUFAs, including linoleic acid, that excess dilutes the effective concentration of 11,11-D2-linoleic acid. In turn, this impacts the amount of 13,13-D2-arachidonic acid that is hepatically generated when all other factors remain constant.

When the amount of total PUF As consumed is such that the amount of 13,13-D2-arachidonic acid hepatically generated is less than about 70% per day of that generated when the average amount of PUFAs is consumed, then that patient is considered to have excessive linoleic acid consumption.

### Pathology

The discovery of several aldehydes that easily reacted with sulfhydryl groups, resulting in the inhibition of vital metabolic processes, led to the association of polyunsaturated fatty acid peroxidation as a component of the pathology of diseases such as ALS (Schauenstein, E.; Esterbauer, H. Formation and properties of reactive aldehydes. Ciba Found. Symp. (67):225-244; 1978). Whether as a primary cause of the disease or a secondary consequence, such lipid peroxidation is attributed to oxidative stress, which leads to neuronal death and such is implicated in the progression of ALS.

The oxidative stress responsible for peroxidation is due to an imbalance between routine production and detoxification of reactive oxygen species (“ROS”) that lead to an oxidative attack on the lipid membrane of cells. The membrane of motor neurons is highly enriched in arachidonic acid. Separating each of these 4 sites are 3 bis-allylic methylene groups and flanking both ends of these 4-sites are mono-allylic methylene groups. The bis-allylic groups are particularly susceptible to oxidative damage due to ROS, and to enzymes such as cyclooxygenases, cytochromes and lipoxygenases, as compared to allylic methylene and methylene groups.

Moreover, once a bis-allylic methylene group in one arachidonic acid is oxidized by a ROS, a cascade of further oxidation of other arachidonic acid groups in the lipid membrane occurs. This is because a single ROS generates oxidation of a first arachidonic acid component through a free radical mechanism which, in turn, can oxidize a neighboring arachidonic acid through the same free radical mechanism which yet again can oxidize another neighboring arachidonic acid in a process referred to as lipid chain auto-oxidation. The resulting damage includes a significant number of oxidized arachidonic acid components in the cell membrane.

Oxidized arachidonic acids negatively affect the fluidity and permeability of cell membranes in motor neurons. In addition, they can lead to oxidation of membrane proteins as well as being converted into a large number of highly reactive carbonyl compounds. The latter include reactive species such as acrolein, malonic dialdehyde, glyoxal, methylglyoxal, etc. (Negre-Salvayre A, et al. Brit. J. Pharmacol. 2008; 153:6-20). But the most prominent products of arachidonic acid oxidation are alpha, beta-unsaturated aldehydes such as 4-hydroxynon-2-enal (4-HNE; formed from n-6 PUFAs like LA or AA), and corresponding ketoaldehydes (Esterfbauer H, et al. Free Rad. Biol. Med. 1991; 11:81-128. As noted above, these reactive carbonyls cross-link (bio)molecules through Michael addition or Schiff base formation pathways which continues the underlying pathology of the disease.

### Disease Progression

When a patient is first diagnosed with ALS, the clinician evaluates that patient's rate of disease progression by assessing the patient's loss of functionality in the absence of therapy as described herein. That rate is referred to as the “natural history” of the disease and is typically measured by standardized tests that measure the extent of a patient's functionality over a set period of time. For example, in the case of ALS, there is a standard test referred to as ALSFRS-R which determines the rate of loss of muscle functionality at a given point in time. Serial testing done over time provides for a measure disease progression. This test has 12 components each of which are measured on a 0 (worse) to 4 (best) scale. Patients with a natural history score of 39 or more in this test are deemed to be in the incipient stage of ALS whereas patients with a natural history score or a subsequent score of 37 or less are deemed to have transitioned into the accelerated stage of ALS.

Heretofore, the treatment of ALS employed deuterated 11,11-D2-linoleic acid or an ester thereof, including those in a lipid bilayer form, to stabilize polyunsaturated fatty acids against ROS. Examples of such treatments are found in: WO 2011/053870, WO 2012/148946, and WO 2020/102596, each of which is incorporated herein by reference in its entirety. Needless to say, the art did not appreciate or suggest that patients with ALS should be treated differently depending on whether the disease was in its incipient or accelerated stage.

As to 11,11-D2-linoleic acid or an ester thereof, this compound acts as a prodrug for 13,13-D2-arachidonic acid-the therapeutic entity. Each of these documents discloses the in vivo conversion of a portion of 11,11-D2-linoleic acid to 13,13-D2-arachidonic acid which is then incorporated into the motor neurons to stabilize these neurons from oxidative damage. The in vivo accumulation of 13,13-D2-arachidonic acid occurs over months until a therapeutic concentration is achieved. Once a therapeutic concentration of 13,13-D2-arachidonic acids is achieved, continued administration of 11,11-D2-linoleic acid or ester thereof is necessary to maintain such a therapeutic concentration.

Still further, the dosing regimen employed must address the patient's need to promptly establish a therapeutic concentration in vivo. Such would protect patients when they transition from the incipient stage to the accelerated stage of the diseases their neural cell membrane is stabilized against LPO. Accordingly, a dosing regimen suitable for the incipient stage of ALS is employed (the “incipient dosing regimen”) and is addressed below.

When deuterated arachidonic acid or ester thereof administered to the patient is 7,7,10,10,13,13-D6-arachidonic acid or an ester thereof, the incipient dosing regimen employed takes into account that this drug or prodrug (in the case of the ester) does not need to be converted in vivo.

### Compound Preparation

11,11-D2-linoleic acid is known in the art and is commercially available. In addition, 11,11-D2-linoleic acid and esters thereof are described, for example, in U.S. Pat. No. 10,052,299 which is incorporated herein by reference in its entirety. 7,7,10,10,13,13-D6-arachidonic acid is described in U.S. Pat. No. 10,730,821 which is incorporated herein by reference in its entirety. Likewise, compositions of 7,7,10,10,13,13-D6-arachidonic acid that comprise, on average, about 80% of the hydrogen atoms at each of the bis-allylic sites having been replaced by deuterium atoms and, on average, no more than about 35% of the hydrogen atoms at the mono-allylic sites having been replaced by deuterium atoms are also disclosed in U.S. Pat. No. 10,730,821.

### Methodology-11,11-D2-Linoleic Acid or Ester Thereof

In one embodiment, the methods described herein utilize in vivo conversion of linoleic acid to arachidonic acid by administering 11,11-D2-linoleic acid or an ester thereof to a patient in order to biosynthesize a therapeutic concentration of 13,13-D2-arachidonic acid for use in the methods described herein.

In one embodiment, an incipient dosing regimen of 11,11-D2-linoleic acid or ester thereof is administered to the patient in sufficient amounts to generate a steady state concentration in red blood cells of at least about 20% based on the total amount of arachidonic acid, including deuterated arachidonic acid, found therein. In one embodiment, the incipient dosing regimen is set to obtain this steady state concentration within about 8 weeks from onset of therapy or earlier (e.g., 6 weeks or 4 weeks). When a steady state concentration is achieved in red blood cells, the attending clinician associated such to a steady state concentration in the neurons.

In one embodiment, the incipient dosing regimen employs an ester of 11,11-D2-linoleic acid such as linoleic acid ethyl ester. Generally, the incipient dosing regimen employs from about 5 to 10 grams of this ester per day and preferably about 9 grams per day. The deuterated linoleic acid ester is preferably administered in three partial doses of 3 grams per day generally with breakfast, lunch and dinner. Periodically, the clinician can ascertain the extent of conversion of this ester into 13,13-D2-arachidonic acid by testing red blood cells as described in in International Patent Application No. PCT/US2022/15368 which is incorporated herein by reference in its entirety.

At the discretion of the attending clinician, once a steady state concentration of at least 20% 13,13-D2-arachidonic acid in red blood cells is achieved (based on the total amount of arachidonic acid in said cell including deuterated arachidonic acid), a lower dose of 11,11-D2-linoleic acid ethyl ester can be administered such as at least about 5 grams per day including about 5 grams or about 6 grams or about 7 grams per day provided that the concentration of 13,13-D2-arachidonic acid in red blood cells remains above 20%.

If the attending clinician elects to reduce the dosing, then the initial dose of 11,11-D2-linoleic acid ethyl ester is designated as the incipient loading or primer dose and the subsequent reduced dose is referred to as the incipient maintenance dose.

When 11,11-D2-linoleic acid or an ester thereof is administered, a portion of this prodrug is enzymatically converted into the active 13,13-D2-arachidonic acid. However, this conversion is typically rate limited thereby limiting the amount of arachidonic acid that the body can enzymatically generate in a given day. As such, only a fraction of the linoleic acid consumed is converted to arachidonic acid with the majority of the linoleic acid including deuterated linoleic acid remaining unchanged. One factor affecting the amount of 13,13-D2-arachidonic acid that can be generated in vivo is the amount of fat consumed by the patient and especially the amount of linoleic acid consumed. Simply mass balance dictates that the more linoleic acid included in the patient's diet coupled with a specific amount of 11,11-D2-linoleic acid or ester thereof correlates to a reduction in the amount of 13,13-D2-arachidonic acid so generated. Accordingly, patients that evidence reduced rates of conversion can be placed on a diet restricted in the amount of linoleic acid consumed.

### Methodology-7,7,10,10,13,13-D6 Arachidonic Acid or Ester Thereof

Unlike linoleic acid, arachidonic acid or an ester thereof does not entail any in vivo conversion other than hydrolysis of the ester to the corresponding acid. As such, arachidonic acid is immediately available to the body for systemic uptake. As noted previously, only about 10% of linoleic acid is enzymatically converted to arachidonic acid. Since arachidonic acid does not require a similar conversion, the dose of 7,7,10,10,13,13-D6-arachidonic acid needs to be only 1/10 that of 11,11-D2-linoleic acid or about 864 milligrams per day. In addition, and as shown in the Examples, 7,7,10,10, 13,13-D6-arachidonic acid is a little more than twice as active in an inflammation model as is 13,13-D2-arachidonic acid. Using this activity differential, then a little less than 430 mg of 7,7,10,10,13,13-D6-arachidonic acid would be the equivalent of 8.64 grams of 11,11-D2-linoleic acid. Recognizing that the incipient dosing amount for 11,11-D2-linoleic acid can vary from as little as 5 grams per day to as much as 10 grams per day, the corresponding variance for 7,7,10,10,13,13-D6-arachidonic acid can be as low as about 250 mg per day to about 500 mg per day. In view of the estimates made herein, a more realistic range for the incipient dosing of 7,7,10,10,13,13-D6-arachidonic acid is from about 100 mg per day to about 2 grams per day.

### EXAMPLES

The methods described herein are further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims. In these examples, the following terms are used herein and have the following meanings. If not defined, the abbreviation has its conventional medical meaning.

### Example 1—Minimal Rate of Loss of Functionality for Patients in the Incipient Stage of ALS

In this example, 41 patients diagnosed with ALS were tested to ascertain their natural history. Of these 41 patients, 23 scored above 38 points in the ALSFRS-R scoring test and were assigned as having incipient ALS. Subsequently, these 23 patients were separated into a first cohort of 13 patients each treated with about 8.64 grams of 11,11-D2-linoleic acid (administered as about 9 grams of the corresponding ethyl ester) and 10 patients treated with placebo. Each cohort received three 1 gram pills t.i.d. (“.three times a day”) at breakfast, lunch and dinner for a total of 9 pills.

Blood draws were taken periodically to assess the concentration of 13,13-D2-arachidonic acid in red blood cells. By week 8, the patients treated with 11,11-D2-linoleic acid ethyl ester evidenced a steady state concentration of 13,13-D2-arachidonic acid ranging from 8-15% based on the total amount of arachidonic acid including deuterated arachidonic acid.

After 24 weeks, each patient in each cohort were retested using the ALSFRS-R scoring test. The scores were then averaged for each cohort and the results are found in FIG. 1. These results evidence that the treated and the untreated cohort experienced minimal loss of functionality with almost identical scores at 24 weeks. Taken together, this suggests that patients in the incipient stage of ALS are agnostic to treatment because their loss of muscular functionality has not reached the accelerated stage of this disease.

After retesting, 8 patients previously treated with placebo and 10 patients previously treated with about 8.64 grams of 11,11-D2-linoleic acid were each given 8.64 grams of 11,11-D2-linoleic acid (administered as about 9 grams of the corresponding ethyl ester) for an additional 24 weeks. Each cohort received three 1 gram pills t.i.d. (“.three times a day”) at breakfast, lunch and dinner for a total of 9 pills.

After 48 weeks, each patient in each cohort were retested using the ALSFRS-R scoring test. The scores were then averaged. Using a cut-off of −11 for change from baseline (based on an approximately −1 point per month or −12 point per year which is mentioned as the typical rate of decline in the ALS world), 70% (7/10) of patients treated 8.64 grams of 11,11-D2-linoleic acid for 48 weeks showed a better rate of decline while only 40% (4/8) patients of the patients treated 8.64 grams of 11,11-D2-linoleic acid for 24 weeks showed a better rate of decline.

### Example 2-Comparative to Patients in the Accelerated Stage of ALS

In this example, the same 41 patients diagnosed with ALS used in Comparative Example 1 were used. However, in this example, 12 patients were assigned as being in the accelerated stage of the disease as each had an ALSFRS-R score of less than 37. Subsequently, these 12 patients were separated into a first cohort of 7 patients each treated with 8.64 grams of 11,11-D2-linoleic acid (administered as 9 grams of the corresponding ethyl ester) and 5 patients treated with placebo. Each cohort received three 1 gram pills t.i.d. at breakfast, lunch and dinner for a total of 9 pills.

Blood draws were taken periodically to assess the concentration of 13,13-D2-arachidonic acid in red blood cells. By week 8, the patients treated with 11,11-D2-linoleic acid ethyl ester evidenced a steady state concentration of 13,13-D2-arachidonic acid ranging from 5-17% based on the total amount of arachidonic acid including deuterated arachidonic acid.

After 24 weeks, each patient in each cohort were retested using the ALSFRS-R scoring test. The scores were then averaged for each cohort and the results are found in FIG. 2. These results evidence that the untreated cohort experienced substantial loss of functionality while the treated cohort exhibited substantially less loss of muscular functionality. Taken together, this suggests that the untreated patients in the accelerate stage of ALS rapidly experience loss of muscular functionality characteristic of this stage of the disease.

### Example 3

This example provides a contemplated result of treating patients with incipient ALS during the incipient stage of this disease and having an average ALSFRS-R score of 42. In this example, a first cohort of these patients are treated with a 11,11-D2-linoleic acid ester at 8.64 grams per day (based on the weight of the de-esterified 11,11-D2-linoleic acid) from the onset of the disease and for 48 weeks. Likewise, a second cohort of these patients is treated with placebo during this period. Based on the results of Comparative Example 1, it is contemplated that after 24 weeks, these patients in both cohorts will have an average ALSFRS-R of about 40. Periodic testing of the patients in both cohorts is continued from week 25 to week 48.

FIG. 3A illustrates contemplated results between the treated group and the placebo group. Here, the underlying therapy extends the period before meaningful loss of functionality begins by several weeks and also provides for improved retention of muscular functionality from week 25 to week 28 by 7 points.

Likewise, FIG. 3B illustrates contemplated results evidencing that the delay in loss of functionality in the treated cohort as compared to placebo is about 4 months.

What is claimed is:

1. A method to delay the onset of or attenuate the rate of loss of muscular functionality during the accelerated stage of amyotrophic lateral sclerosis (ALS) which stage is characterized by the rapid loss of muscular functionality in patients the method comprising:
(a) ascertaining a current level of muscular functionality in a patient prior to initiating treatment by generating a natural history score using the ALSFRS-R scoring system, wherein the patient is identified as having incipient ALS when the patient scores at least 39 points out of a total of 48 possible points; and
(b) initiating treatment by periodically administering to the patient identified as being in the incipient stage of ALS with a prophylactic dosing regimen of a deuterated arachidonic acid or a prodrug thereof thereby delaying the onset of or attenuating the rate of loss of muscular functionality during the accelerated stage of the disease.

2. The method of claim 1, wherein said deuterated arachidonic acid or a prodrug thereof is 11,11-D2-linoleic acid or an ester thereof.

3. The method of claim 2, wherein said 11,11-D2-linoleic acid or an ester thereof is an ester.

4. The method of claim 3, wherein said 11,11-D2-linoleic acid ester is an ethyl ester.

5. The method of claim 4, wherein said 11,11-D2-linoleic acid ethyl ester is administered periodically to the patient at a dose of about 5 to about 10 grams/day.

6. The method of claim 5, wherein the extent that 13,13-D2-arachidonic acid enzymatically generated in vivo from said 11,11-D2-linoleic acid ethyl ester is assessed in each patient by measuring the concentration of 13,13-D2-arachidonic acid in red blood cells.

7. The method of claim 1, wherein said deuterated arachidonic acid or a prodrug thereof is 7,7, 10, 10, 13, 13-D6-arachidonic acid or an ester thereof.

8. The method of claim 7, wherein said 7,7,10,10,13,13-D6-arachidonic acid or an ester thereof is an ester.

9. The method of claim 8, wherein said 7,7,10,10,13,13-D6-arachidonic acid ester is an ethyl ester.

10. The method of claim 9, wherein said 7,7,10,10,13,13-D6-arachidonic acid ethyl ester is administered periodically to the patient at a dose of about 5 to about 10 grams/day.

11. The method of claim 10, wherein the extent that 7,7,10,10,13,13-D6-arachidonic acid absorbed in vivo is assessed in each patient by measuring the concentration of 7,7,10,10,13,13-D6-arachidonic acid in red blood cells.
